(19) United States
(12) Patent Application Publication
(10) Pub. No.: US 2008/0146820 A1
(43) Pub. Date: Jun. 19, 2008

(54) Phosphate fluorosurfactant and siloxane surfactant

(76) Inventors: Axel Hans-Joachim Herzog, West Chester, PA (US); Shaun Raymond Rinehimer, Philadelphia, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 11/983,838

(22) Filed: Nov. 13, 2007

Related U.S. Application Data
(60) Provisional application No. 60/875,068, filed on Dec. 15, 2006.

(51) Int. Cl. C07D 521/00; C07F 9/02

(52) U.S. Cl. 549/214; 568/8

(57) ABSTRACT

This invention relates to surfactant compositions comprising a phosphate fluorosurfactant and a siloxane surfactant wherein the surfactant composition's ability to lower equilibrium surface tension exceeds that of either the phosphate fluorosurfactant or the siloxane surfactant. Because of the synergistic interaction of the phosphate fluorosurfactant and the siloxane surfactant, it is possible to use only small amounts of the more expensive fluorosurfactant in the surfactant composition of the present invention while preserving or improving the ability to lower the equilibrium surface tension of a liquid, such as water.

### FIELD OF THE INVENTION

This invention relates to surfactant compositions comprising a phosphate fluorosurfactant and a siloxane surfactant wherein the surfactant composition's ability to lower equilibrium surface tension exceeds that of either the phosphate fluorosurfactant or the siloxane surfactant.

### BACKGROUND OF THE INVENTION

Surfactants lower the surface tension of a liquid thereby imparting improved surface effects such as spreading, wettability, penetrability, foam inhibition and dispersibility. These improved surface effects are advantageous in many industrial applications including aqueous coatings such as inks, paints, varnishes, and the like.

Equilibrium surface tension refers to the surface tension measured after the liquid and added surfactants have reached equilibrium. Liquids with poor (high) equilibrium surface tension may initially spread smoothly and evenly across a surface but after some time will “de-wet” resulting in undesirable surface defects. This occurs because the passage of time allows the liquid and added surfactant to reach an equilibrium surface tension which is undesirably high thereby causing a liquid which was initially smooth and evenly spread to “crawl back” or “retract” from the surface thereby creating an uneven and rippled spreading. Poor equilibrium surface tension is particularly detrimental in paints which are expected to dry as a smooth and even coating.

Two commonly used classes of surfactants for lowering equilibrium surface tension are fluorosurfactants and siloxane surfactants. Fluorosurfactants typically impart lower equilibrium surface tension compared to siloxane surfactants. However, because fluorosurfactants are typically higher in cost compared with siloxane surfactants, fluorosurfactants are often mixed with less expensive siloxane surfactants to produce less costly surfactant compositions. For example, U.S. Pat. No. 5,852,075 discloses a surfactant composition comprising a fluorosurfactant and a siloxane surfactant.

U.S. Pat. No. 5,852,075, as well as many other relevant documents, lacks any teaching of surfactant compositions comprising a fluorosurfactant and a siloxane surfactant wherein the surfactant composition's ability to lower equilibrium surfactant tension exceeds that of its individual components. When fluorosurfactants are combined with siloxane surfactants it is expected that the resulting surfactant composition would have an ability to lower surface tension which exceeds the siloxane surfactant but does not exceed the fluorosurfactant.

### BRIEF SUMMARY OF THE INVENTION

The present invention identifies specific fluorosurfactants and siloxane surfactants that combine to produce surfactant compositions wherein the surfactant composition's ability to lower equilibrium surface tension exceeds that of either the fluorosurfactant or the siloxane surfactant. It has now been discovered that a phosphate fluorosurfactant synergistically interacts with a siloxane surfactant in the present invention. Specifically, the present invention describes a mixture comprising phosphate fluorosurfactant and siloxane which imparts lower equilibrium surface tension compared to the separate individual components of the mixture. In addition to a blend of individual components, the term “mixture” is also intended to include any product which may result from the reaction or other interaction of the individual components.

Phosphate fluorosurfactants useful for creating a synergistic effect when combined with the siloxane surfactants of the present invention is represented by Formula 1 as follows:

[CₘF₂ₘ₊₁CₙH₂ₙ—O]_{y}P(O)(OM)_{3-y} Formula 1

wherein

M is H, alkali metal ammonium, or NR¹R²R³ wherein each of R¹, R² and R³ are independently H, C₁ to C₂₀ alkyl, or C₁ to C₂₀ hydroxyalkyl,

m is an integer from 4 to 12,

n is an integer from 1 to 16,

y is a number of average value from 1.0 to 2.5,

provided that the two radicals Cₘ and Cₙ contain jointly a straight chain of not less than 8 carbon atoms.

The phosphate fluorosurfactant is preferably a phosphate ester, and is more preferably a salt of a phosphate ester.

Siloxane surfactants useful for creating a synergistic effect when combined with the fluorosurfactants of the present invention are represented by the general Formulae 2A, 2B, 2C, or 3 as follows:

(R²)₃SiO[Si(R²)₂O]_{y}[Si(R²)(R¹)O]ₓ[Si(R²)₂O]_{z}Si(R²)₃ Formula 2A

(R²)₃SiO[Si(R²)₂O]ₓSi(R²)₂R¹ Formula 2B

R¹(R²)₂SiO[Si(R²)₂O]ₓSi(R²)₂R¹ Formula 2C

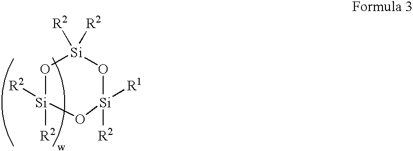

wherein

each R² is independently H, alkyl, or aryl;

each R¹ is a polyoxyalkylene group having the formula 4 as follows:

—CₙR⁴ₚH₂ₙ₋ₚQCₘR⁵ₚH₂ₘ₋ₚOZR³ Formula 4

wherein

each R⁴ and R⁵ is independently H, alkyl, or aryl;

Q is CₙHR⁴, aryl, CH₂CH(OR⁴), CH₂(CH₂OR⁴), S, O, SO, SO₂, SO₂NR⁴, OC(O), OC(NR⁴), NHC(X)NH, or OC(X)NH or triazole;

Z is [C₂H₄O]ₐ and [C₃H₆O]_{b} in block or random order;

X is O or S;

m and n are each independently an integer of 2 to 8;

a is an integer of 0 to about 30; b is an integer of 0 to about 20;

provided that a+b is from 1 to about 50;

each R³ is H, acyl, or a linear or branched alkyl or aryl group having 1 to about 20 carbon atoms;

w is an integer of 1 to 3;

x is an integer of from 1 to about 20;

y is an integer of from 0 to about 20; and

z is an integer of from 0 to about 10.

Preferably R² is H, CH₃, C₂H₅, or C₆H₅; more preferably H or CH₃; and most preferably CH₃. In a particularly preferable embodiment of the invention, each R¹ is a polyoxyalkylene group having the Formula 4 where p is 1, Q is 0, m is 2, p is 1, R⁵ is H, a is 7, b is 0, and R³ is H.

Because of the synergistic interaction of the phosphate fluorosurfactant and the siloxane surfactant, it is possible to use only small amounts of the more expensive fluorosurfactant in the surfactant composition of the present invention while preserving or improving the ability to lower the equilibrium surface tension of a liquid, such as water. For example, the surfactant compositions of the present invention can comprise a mixture of phosphate fluorosurfactant and siloxane surfactant wherein the amount of phosphate fluorosurfactant in the mixture is no more than 35 weight percent, or no more than 21 weight percent, or no more than 18 weight percent, or no more than 10 weight percent.

The present invention also contemplates a surfactant composition consisting essentially of a mixture of the aforementioned phosphate fluorosurfactant and siloxane surfactant wherein the surfactant composition preferably has no other ingredient in excess of 10 weight percent, preferably 5 weight percent, more preferably 1 weight percent, and more preferably 0.5 weight percent.

Because of the synergistic interaction of the phosphate fluorosurfactant and the siloxane surfactant, it is possible to use only small amounts of the resulting surfactant composition to adequately lower equilibrium surface tension. For example, the present invention includes an aqueous solution, dispersion, or emulsion comprising between 0.01 to 2.0 weight percent of the aforementioned surfactant composition and optionally having lower amounts such as between 0.01 to 1.0 weight percent, or between 0.01 and 0.1 weight percent.

### BRIEF DESCRIPTION OF THE DRAWING(S)

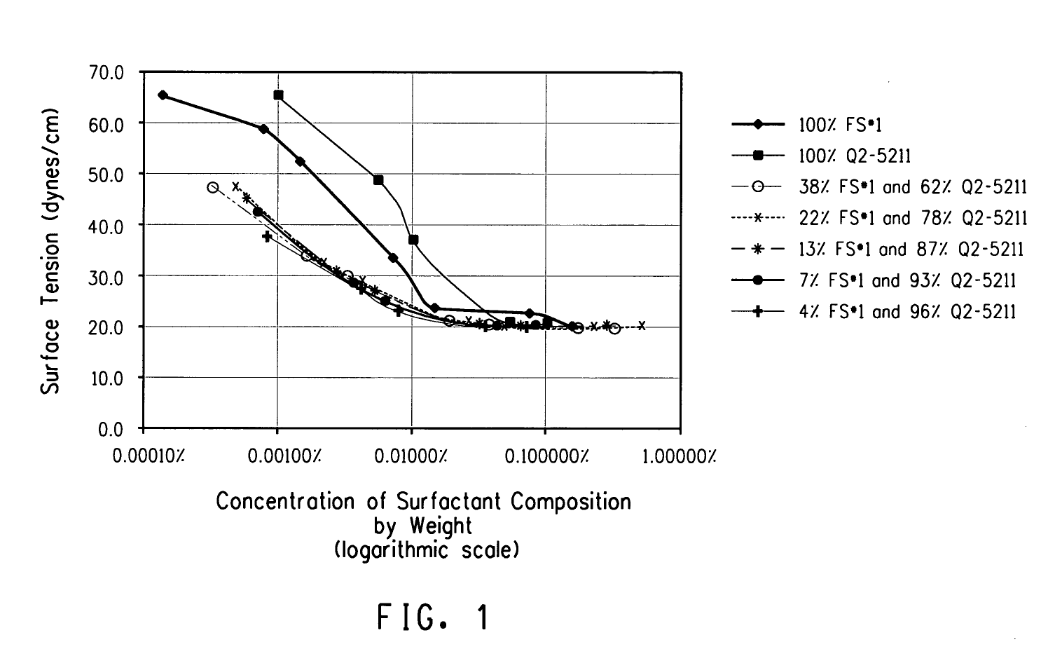

As described further herein, FIGS. 1, 2 and 3 are graphical representations of concentration versus equilibrium surface tension for surfactant compositions of the present invention as well as comparative surfactant compositions.

### DETAILED DESCRIPTION OF THE INVENTION

The present invention describes a mixture comprising phosphate fluorosurfactant and siloxane which imparts lower equilibrium surface tension compared to the separate individual components of the mixture. In addition to a blend of individual components, the term “mixture” is also intended to include any product which may result from the reaction or other interaction of the individual components.

Phosphate fluorosurfactants useful for creating a synergistic effect when combined with the siloxane surfactants of the present invention is represented by Formula 1 as follows:

[CₘF₂ₘ₊₁CₙH₂ₙ—O]_{y}P(O) (OM)_{3-y} Formula 1

wherein

M is H, alkali metal ammonium, or NR¹R²R³ wherein each of R¹, R² and R³ are independently H, C₁ to C₂₀ alkyl, or C₁ to C₂₀ hydroxyalkyl,

m is an integer from 4 to 12,

n is an integer from 1 to 16,

y is a number of average value from 1.0 to 2.5,

provided that the two radicals Cₘ and Cₙ contain jointly a straight chain of not less than 8 carbon atoms.

The phosphates of Formula 2 are prepared by reacting the corresponding polyfluoroalkanol with phosphorus oxychloride in the presence of an acid acceptor such as pyrridine, or with phosphorus pentoxide, in the optional presence of an organic liquid diluent such as benzene, toluene, or xylene. Further details are provided in U.S. Pat. No. 3,083,224.

Preferred phosphate fluorosurfactants are phosphate esters containing a perfluoroalkyl group, or mixtures and salts thereof. Such polyfluoroalkyl phosphate esters are prepared using conventional techniques. For example, a hydroperfluoroalkanol is reacted with phosphoric anhydride to provide a mixture of perfluoroalkyl phosphate and pyrophosphate esters. This mixture is reacted with a glycol, such as ethylene glycol, to convert the pyrophosphate esters to to mono-perfluoroalkylphosphate esters and bis-perfluoroalkyl/ethylene glycol phosphate esters. The mixture is then neutralized with a base such as ammonium hydroxide to obtain the phosphate ester salts. Further details on the preparation of these materials is contained in U.S. Pat. No. 3,083,224.

Siloxane surfactants useful for creating a synergistic effect when combined with the fluorosurfactants of the present invention are represented by the general Formulae 2A, 2B, 2C, or 3 as follows:

(R²)₃SiO[Si(R²)₂O]_{y}[Si(R²)(R¹)O]ₓ[Si(R²)₂O]_{z}Si(R²)₃ Formula 2A

(R²)₃SiO[Si(R²)₂O]ₓSi(R²)₂R¹ Formula 2B

R¹(R²)₂SiO[Si(R²)₂O]ₓSi(R²)₂R¹ Formula 2C

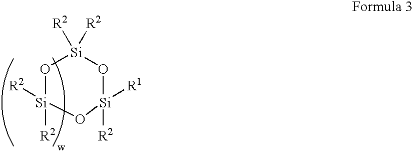

wherein
each R² is independently H, alkyl, or aryl;
each R¹ is a polyoxyalkylene group having the formula 4 as follows:

—CₙR⁴ₚH₂ₙ₋ₚQCₘR⁵ₚH₂ₘ₋ₚOZR³ Formula 4

wherein

each R⁴ and R⁵ is independently H, alkyl, or aryl;

Q is CNHR⁴, aryl, CH₂CH(OR⁴), CH₂(CH₂OR⁴), S, O, SO, SO₂, SO₂NR⁴, OC(O), OC(NR⁴), NHC(X)NH, or OC(X)NH or triazole;

Z is [C₂H₄O]ₐ and [C₃H₆O]_{b} in block or random order;

X is O or S;

m and n are each independently an integer of 2 to 8;

a is an integer of 0 to about 30; b is an integer of 0 to about 20;

provided that a+b is from 1 to about 50;

each R³ is H, acyl, or a linear or branched alkyl or aryl group having 1 to about 20 carbon atoms;

w is an integer of 1 to 3;

x is an integer of from 1 to about 20;

y is an integer of from 0 to about 20; and

z is an integer of from 0 to about 10.

Preferably R² is H, CH₃, C₂H₅, or C₆H₅; more preferably H or CH₃; and most preferably CH₃. In a particularly preferable embodiment of the invention, each R¹ is a polyoxyalkylene group having the Formula 4 where p is 1, Q is 0, m is 2, p is 1, R⁵ is H, a is 7, b is 0, and R³ is H.

Many of siloxane surfactants suitable for use in the invention are commercially available including Q2-5211 available from Dow Corning Corporation, Midland, Mich.; and SILWET L7608 available from available from GE Silicones General Electric Company, Wilton, Conn.

The compounds of Formula 2A, 2B, 2C, and 3 may be prepared as follows. The Q-containing species are synthesized according to common published procedures. A summary of these organic transformation reactions can be found in “Comprehensive Organic Transformations” by Richard C. Larock, Wiley-VCH, New York, N.Y., 2^{nd} Edition, 1999.

Generally the attachment of ω-functionalized alkyl groups to the siloxane is accomplished via hydrosilylation of the corresponding ω-functionalized olefin with a silane moiety containing siloxane. In parallel, the polyoxyalkylenes are terminated with ω-functionalized alkylenes via the reaction of the polyoxyalkylene alkoxides with ω-functionalized α-halides and tosylates, respectively, via nucleophilic substitution reactions. If ω-functionalized alkylenes are pre-reacted with ω-functionalized alpha-halides and tosylates, respectively, via their ω-positioned functions, the resulting α-halides/tosylates-ω-olefine intermediates can be reacted further with the polyoxyalkylene alkoxides and, in turn, the desired siloxane surfactant is obtained upon hydrosilylation of the olefin terminated Q-containing polyoxyalkylene species with a silane containing siloxane. Specifically, CₙHR⁴ and arylene containing linker are obtained using the corresponding olefins terminated polyalkyleneoxide precursors.

Derivatives containing CH₂CH(OR⁴) and CH₂(CH₂OR⁴) are furnished by reaction of a glycidyl terminated polyalkylene glycols with ω-hydroxylalkyl substituted siloxanes or glycidyl terminated siloxane with ω-hydroxylalkyl substituted polyalkylene glycols under acid and basic reaction conditions, respectively, optionally followed by alkylation.

The surfactant compositions of the present invention comprise a mixture of the phosphate fluorosurfactant and the siloxane surfactant described herein. As shown in the examples that follow, because of the synergistic interaction with the siloxane surfactant, it has been discovered that only a small amount of the phosphate fluorosurfactant of the present invention is required to improve the resulting surfactant composition's ability to lower equilibrium surface tension. For example, no more than 35 weight percent of the phosphate fluorosurfactant of the present invention is required to improve the resulting surfactant composition's ability to lower equilibrium surface tension. It has further been discovered that amounts of phosphate fluorosurfactant much lower than 35 weight percent maintain the ability to improve the resulting surfactant composition's ability to lower equilibrium surface tension. Accordingly, the surfactant compositions of the present invention can have an amount of phosphate fluorosurfactant of no more than 21 weight percent, or no more than 15 weight percent, or no more than 10 weight percent.

The amount of siloxane surfactant in the surfactant compositions of the present invention depends upon the desired amount of phosphate fluorosurfactant therein. Because of synergistic interaction with the siloxane surfactant, it has been discovered that only a small amount of the phosphate fluorosurfactant of the present invention is required to improve the resulting surfactant composition's ability to lower equilibrium surface tension. Consequently, the surfactant compositions of the present invention comprise mixtures with small amounts of the relatively costly fluorosurfactant and large amounts of the relatively inexpensive siloxane surfactant. Examples include surfactant compositions comprising mixtures of phosphate fluorosurfactant with greater than 79 weight percent of the siloxane surfactant, preferably greater than 85 weight percent, more preferably greater than 90 weight percent, and most preferably greater than 5 weight percent.

It is preferable that the surfactant compositions of the present invention are essentially comprised of the phosphate fluorosurfactant and siloxane surfactant described herein. Even more preferably, the surfactant are essentially comprised of the phosphate fluorosurfactant and siloxane surfactant described herein such that preferably no more than 10 weight percent of any other ingredient is present, more preferably no more than 5 weight percent of any other ingredient is present, and even more preferably no more than 1 weight percent of any other ingredient is present, and most preferably no more than 0 weight percent of any other ingredient is present.

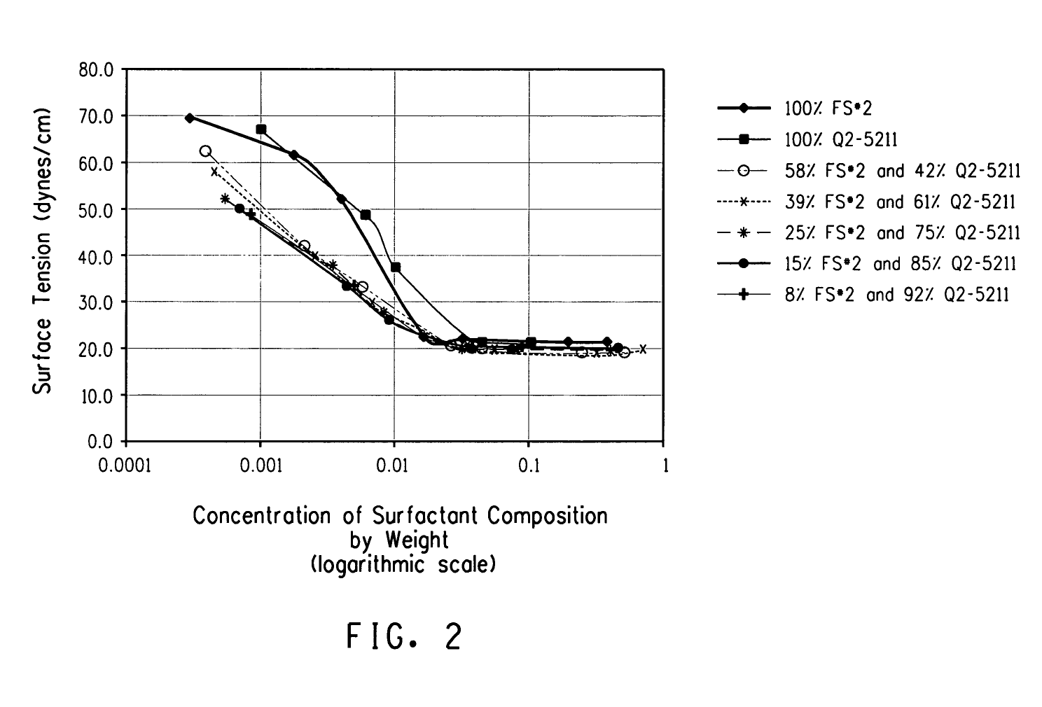

The surfactant compositions of the present invention can be added to any virtually any liquid to reduce the equilibrium surface tension thereof. The surfactant compositions of the present invention are particularly suited for use in aqueous solutions, dispersions, or emulsions. Because of the synergistic interaction of the components thereof, only small amounts of the surfactant composition of the present invention are required to lower equilibrium surface tension. The amount of surfactant composition of the invention added can be as low as 2 weight percent based on the weight of the liquid. As shown by FIGS. 1 and 2, the amount of surfactant composition added can be as low as 1, 0.1, or even 0.01 weight percent based on the weight of the liquid.

### EXAMPLES

### Fluorosurfactants

FS#1 and FS#2 are different fluoroalkyl phosphate ammonium salts which are mixed with a glycol ester, available from E.I. du Pont de Nemours and Company, Wilmington. The fluoroalkyl phosphate ammonium salt in FS#1 has a longer perfluoroalkyl chain compared to FS#2. FS#3 is a fluorosurfactant having no phosphate group and is more specifically a fluoroalkylethoxylate prepared as described in U.S. Pat. No. 5,567,857, and available from E.I. du Pont de Nemours and Company, Wilmington, Del.

### Siloxane Surfactant

For all of the examples below, the siloxane surfactant used was a trisiloxane ethoxylate and commercially available as Q2-5211 from Dow Corning Corporation. Midland, Mich.

### Surface Tension Measurements

In examples 1-4, equilibrium surface tension was measured in accordance with the following procedure. An aliquot (30 mL) of each aqueous solution was poured into separate glass dishes and allowed to equilibrate for 20-30 seconds before measurements were taken. The measurements were provided using a Krüss K11 tensiometer (available from Krüss GmbH, Hamburg, Germany) using the ‘Wilhelmy Plate Method’ wherein a small platinum plate with a roughened surface is suspended perpendicular to the liquid surface contained in the glass dish. The plate is attached to a force measuring balance. The glass dish is raised manually until the surface of the liquid is a few millimeters in distance from the suspended plate. The dish is then raised electronically and the wetting of the plate provides for a force proportional to the surface tension of the liquid. A mean surface tension value was obtained from ten consecutive readings and reported in units of dyne/cm where 1 dyne/cm is equivalent to 1 mN/M. A mean equilibrium surface tension value for each dilution is shown herein on column 2 of Table 1 and on column 3 of Tables 2, 3, and 4. In certain cases, marked by an asterisk (*) on the tables, it was not possible to obtain a surface tension measurement because a homogenous solution was not achieved.

### Example 1 (Comparative)

In this comparative example, a surfactant composition was made with no fluorosurfactant and made only with siloxane surfactant, specifically Q2-5211. Aqueous solution of Q2-5211 dissolved in weight percents listed on column 1 of Table 1 were prepared and stirred for a period of 18-24 hours. Equilibrium surface tension measurements were taken of each of the aqueous solution and are shown on column 2 of Table 1. A graphical representation of concentration versus equilibrium surface tension of these aqueous solutions is depicted as square shapes in FIGS. 1, 2, and 3.

TABLE 1

| Concentration of Q2- | |
|---|---|
| 5211 by weight | Surface Tension (dyne/cm) |
| 1 | * |
| 0.5 | * |
| 0.1 | 20.6 |
| 0.05 | 21.0 |
| 0.01 | 37.1 |
| 0.005 | 48.6 |
| 0.001 | 65.3 |

### Example 2

In this example, six surfactant compositions ranging from 100 to 4 weight percent of FS#1 (a phosphate fluorosurfactant) and from 0 to 96 weight percent Q2-5211 were made and are listed in column 1 of Table 2. Seven aqueous solutions of each surfactant composition in decreasing concentrations were prepared and stirred for a period of 18-24 hours. The amount by weight percent of the surfactant composition in aqueous solution is shown on column 2 of Table 2. Equilibrium surface tension measurements were taken of each of the aqueous solution and are shown on column 3 of Table 2. A graphical representation of concentration versus equilibrium surface tension of each of the six surfactant compositions is depicted in FIG. 1.

As shown in FIG. 1, surfactant compositions which are a mixture of FS#1 and Q2-5211 are all superior to surfactant compositions containing only 100% FS#1 or only 100% Q2-5211. Accordingly, FIG. 1 shows synergy, i.e. a mixture comprising phosphate fluorosurfactant and siloxane surfactant imparts lower equilibrium surface tension compared to the separate individual components of the mixture.

TABLE 2

| Components of | | |
|---|---|---|
| Surfactant | Concentration of | |
| Composition by | Surfactant Composition | Surface Tension |
| Weight | by Weight | (dyne/cm) |
| 100% FS#1 | 0.15 | 20.6 |
| | 0.075 | 22.6 |
| | 0.015 | 24.3 |
| | 0.0075 | 33.6 |
| | 0.0015 | 52.6 |
| | 0.00075 | 58.9 |
| | 0.00015 | 65.6 |
| 38% FS#1 | 0.32 | 19.5 |
| | 0.16 | 19.6 |
| | 0.032 | 20.5 |
| | 0.016 | 21.0 |
| | 0.0032 | 28.3 |
| | 0.0016 | 33.8 |
| | 0.00032 | 47.7 |
| 22% FS#1 | 0.448 | 19.7 |
| 78% Q2-5211 | 0.224 | 19.8 |
| | 0.0448 | 20.0 |
| | 0.0224 | 20.9 |
| | 0.00448 | 27.9 |
| | 0.00224 | 32.8 |
| | 0.000448 | 46.8 |
| 13% FS#1 | 0.575 | * |
| 87% Q2-5211 | 0.2875 | 20.0 |
| | 0.0575 | 20.7 |
| | 0.02875 | 20.8 |
| | 0.00575 | 26.0 |
| | 0.002875 | 30.2 |
| | 0.000575 | 43.8 |
| 7% FS#1 | 0.703 | * |
| 93% Q2-5211 | 0.3515 | * |
| | 0.0703 | 20.5 |
| | 0.03515 | 20.6 |
| | 0.00703 | 24.0 |
| | 0.003515 | 28.7 |
| | 0.000703 | 41.5 |
| 4% FS#1 | 0.83 | * |
| | 0.415 | * |
| | 0.083 | 20.4 |
| | 0.0415 | 20.6 |
| | 0.0083 | 22.5 |
| | 0.00415 | 27.0 |
| | 0.00083 | 37.2 |

### Example 3

In this example, six surfactant compositions ranging from 100 to 8 weight percent of FS#2 (a phosphate fluorosurfactant) and from 0 to 92 weight percent Q2-5211 were made and are listed in column 1 of Table 3. Seven aqueous solutions of each surfactant composition in decreasing concentrations were prepared and stirred for a period of 18-24 hours. The amount by weight percent of the surfactant composition in aqueous solution is shown on column 2 of Table 3. Equilibrium surface tension measurements were taken of each of the aqueous solution and are shown on column 3 of Table 3. A graphical representation of concentration versus equilibrium surface tension of each of the six surfactant compositions is depicted in FIG. 2.

As shown in FIG. 2, surfactant compositions which are a mixture of FS#2 and Q2-5211 are all superior to surfactant compositions containing only 100% FS#1 or only 100% Q2-5211. Accordingly, FIG. 2 shows synergy, i.e. a mixture comprising phosphate fluorosurfactant and siloxane surfactant imparts lower equilibrium surface tension compared to the separate individual components of the mixture.

TABLE 3

| Components of | | |
|---|---|---|
| Surfactant | Concentration of | |
| Composition by | Surfactant Composition | Surface Tension |
| Weight | by Weight | (dyne/cm) |
| 100% FS#2 | 0.34 | 20.8 |
| | 0.170 | 21.2 |
| | 0.034 | 22.7 |
| | 0.0170 | 23.1 |
| | 0.0034 | 52.9 |
| | 0.00170 | 61.0 |
| | 0.00034 | 69.4 |
| 58% FS#2 | 0.472 | 18.7 |
| | 0.236 | 18.4 |
| | 0.0472 | 20.0 |
| | 0.0236 | 21.6 |
| | 0.00472 | 34.8 |
| | 0.00236 | 41.3 |
| | 0.000472 | 62.2 |
| 39% FS#2 | 0.571 | 18.8 |
| 61% Q2-5211 | 0.2855 | 18.6 |
| | 0.0571 | 20.3 |
| | 0.02855 | 21.6 |
| | 0.00571 | 30.0 |
| | 0.002855 | 39.5 |
| | 0.000571 | 57.9 |
| 25% FS#2 | 0.67 | * |
| 75% Q2-5211 | 0.355 | 19.6 |
| | 0.067 | 20.4 |
| | 0.0355 | 21.4 |
| | 0.0067 | 28.9 |
| | 0.00355 | 37.3 |
| | 0.00067 | 52.4 |
| 15% FS#2 | 0.769 | * |
| 85% Q2-5211 | 0.3845 | 19.9 |
| | 0.0769 | 20.6 |
| | 0.03845 | 21.4 |
| | 0.00769 | 27.9 |
| | 0.003845 | 34.4 |
| | 0.000769 | 50.1 |
| 8% FS#2 | 0.868 | * |
| | 0.434 | * |
| | 0.0868 | 20.7 |
| | 0.0434 | 21.4 |
| | 0.00868 | 27.6 |
| | 0.00434 | 34.1 |
| | 0.000868 | 49.3 |

### Example 4 (Comparative)

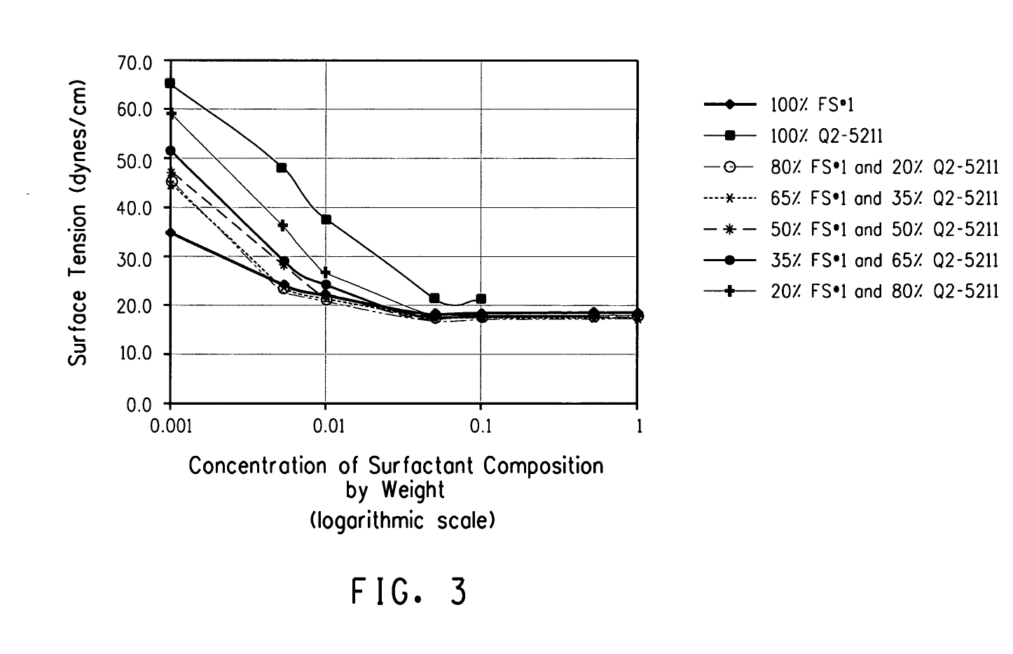

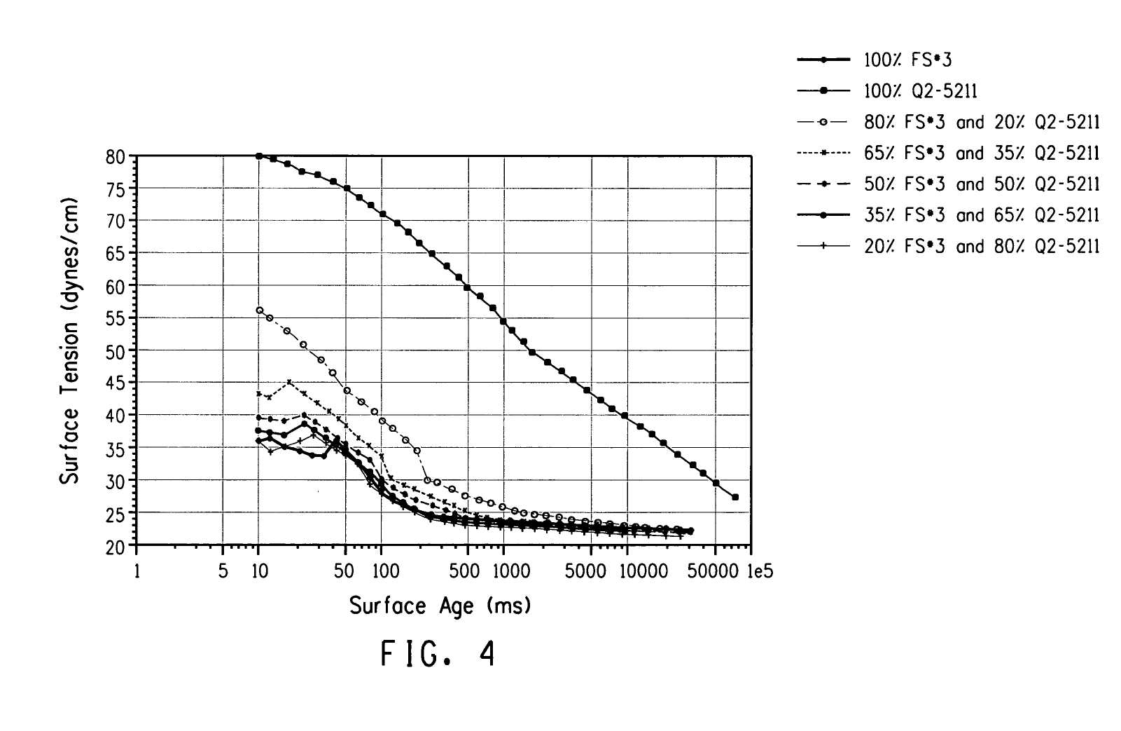

In this comparative example, six surfactant compositions ranging from 100 to 20 weight percent of FS#3 (a fluoroalkylethoxylate surfactant with no phosphate group) and from 0 to 80 weight percent Q2-5211 were made and are listed in column 1 of Table 4. Seven aqueous solutions of each surfactant composition in decreasing concentrations were prepared and stirred for a period of 18-24 hours. The amount by weight percent of the surfactant composition in aqueous solution is shown on column 2 of Table 4. Equilibrium surface tension measurements were taken of each of the aqueous solution and are shown on column 3 of Table 4. A graphical representation of concentration versus equilibrium surface tension of each of the six surfactant compositions is depicted in FIG. 3.

As shown in FIG. 3, surfactant compositions which are a mixture of FS#3 and Q2-5211 are all superior to 100% Q2-5211 but not superior to 100%. Accordingly, FIG. 3 does not show synergy, i.e. a mixture comprising a fluoroalkylethoxylate surfactant and siloxane surfactant does not impart lower equilibrium surface tension compared to at least one of the components of the mixture, in this case, the fluoroalkylethoxylate surfactant. FIG. 3 further shows that not all fluorosurfactants will act synergistically with siloxanes surfactants to lower equilibrium surface tension. However, if the fluorosurfactant has a phosphate group, synergy occurs with the siloxane surfactant with respect to lowering equilibrium surface tension as shown in FIGS. 1 and 2 and described in Examples 2 and 3 herein.

TABLE 4

| Components of | | |
|---|---|---|
| Surfactant | Concentration of | |
| Composition by | Surfactant Composition | Surface Tension |
| Weight | by Weight | (dyne/cm) |
| 100% FS#3 | 1 | 17.8 |
| | 0.5 | 17.9 |
| | 0.1 | 17.9 |
| | 0.05 | 18.2 |
| | 0.01 | 21.2 |
| | 0.005 | 24.6 |
| | 0.001 | 34.5 |
| 80% FS#3 | 1 | 16.8 |
| | 0.5 | 17.0 |
| | 0.1 | 17.3 |
| | 0.05 | 17.5 |
| | 0.01 | 20.8 |
| | 0.005 | 24.7 |
| | 0.001 | 44.8 |
| 65% FS#3 | 1 | 17.1 |
| 35% Q2-5211 | 0.5 | 17.2 |
| | 0.1 | 17.3 |
| | 0.05 | 17.3 |
| | 0.01 | 22.0 |
| | 0.005 | 25.4 |
| | 0.001 | 44.4 |
| 50% FS#3 | 1 | * |
| 50% Q2-5211 | 0.5 | 17.4 |
| | 0.1 | 17.6 |
| | 0.05 | 17.6 |
| | 0.01 | 21.8 |
| | 0.005 | 28.5 |
| | 0.001 | 46.4 |
| 35% FS#3 | 1 | * |
| 65% Q2-5211 | 0.5 | 17.9 |
| | 0.1 | 18.0 |
| | 0.05 | 17.6 |
| | 0.01 | 23.4 |
| | 0.005 | 29.1 |
| | 0.001 | 51.3 |
| 20% FS#3 | 1 | * |
| | 0.5 | * |
| | 0.1 | 18.2 |
| | 0.05 | 18.4 |
| | 0.01 | 26.3 |
| | 0.005 | 35.7 |
| | 0.001 | 59.3 |

What is claimed is:

1. A surfactant composition comprising a mixture of fluorosurfactant and a siloxane wherein:
a) the fluorosurfactant is of the Formula 1
CₘF₂ₘ₊₁CₙH₂ₙ—O]_{y}P(O)(OM)_{3-y} Formula 1
wherein
M is H, alkali metal ammonium, or NR¹R²R³ wherein each of R¹, R² and R³ are independently H, C₁ to C₂₀ alkyl, or C₁ to C₂₀ hydroxyalkyl,
m is an integer from 4 to 12,
n is an integer from 1 to 16,
y is a number of average value from 1.0 to 2.5,
provided that the two radicals Cₘ and Cₙ contain jointly a straight chain of not less than 8 carbon atoms; and
b) the siloxane is of Formulae 2A, 2B, 2C or 3
(R²)₃SiO[Si(R²)₂O]_{y}[Si(R²)(R¹)O]ₓ[Si(R²)₂O]_{z}Si(R²)₃ Formula 2A
(R²)₃SiO[Si(R²)₂O]ₓSi(R²)₂R¹ Formula 2B
R¹(R²)₂SiO[Si(R²)₂O]ₓSi(R²)₂R¹ Formula 2C 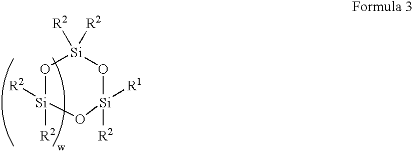
wherein
each R² is independently H, alkyl, or aryl;
each R¹ is a polyoxyalkylene group having the Formula 4 as follows:
—CₙR⁴ₚH₂ₙ₋ₚQCₘR⁵ₚH₂ₘ₋ₚOZR³ Formula 4
wherein
each R⁴ and R⁵ is independently H, alkyl, or aryl;
Q is CₙHR⁴, aryl, CH₂CH(OR⁴), CH₂(CH₂OR⁴), S, O, SO, SO₂, SO₂NR⁴, OC(O), OC(NR⁴), NHC(X)NH, or OC(X)NH or triazole;
Z is [C₂H₄O]ₐ and [C₃H₆O]_{b} in block or random order;
X is O or S;
m and n are each independently an integer of 2 to 8;
a is an integer of 0 to about 30; b is an integer of 0 to about 20; provided that a+b is from 1 to about 50;
each R³ is H, acyl, or a linear or branched alkyl or aryl group having 1 to about 20 carbon atoms;
w is an integer of 1 to 3;
x is an integer of from 1 to about 20;
y is an integer of from 0 to about 20; and
z is an integer of from 0 to about 10.

2. The composition of claim 1 wherein R² is H, CH₃, C₂H₅, or C₆H₅.

3. The composition of claim 1 wherein R¹ is a polyoxyalkylene group having the Formula 4 where p is 1, Q is 0, m is 2, p is 1, R⁵ is H, a is 7, b is 0, and R³ is H.

4. The composition of claim 1 wherein the fluorosurfactant is a phosphate ester.

5. The composition of claim 1 wherein the fluorosurfactant is a salt of a phosphate ester.

6. The composition of claim 1 comprising no more than 35 weight percent of the fluorosurfactant.

7. The composition of claim 1 comprising no more than 21 weight percent of the fluorosurfactant.

8. The composition of claim 1 consisting essentially of the mixture of fluorosurfactant and siloxane and having no other ingredient in excess of 10 weight percent.

9. An aqueous solution, dispersion, or emulsion comprising between 0.01 and 2.0 weight percent of the composition of claim 1.
